(19) United States
(12) Patent Application Publication
(10) Pub. No.: US 2004/0191241 A1
(43) Pub. Date: Sep. 30, 2004

(54) Antigen with haptenic mannitol epitopes and a method of preparing antibodies specific to D-mannitol

(75) Inventors: Yeldur Padmanabha Rao Venkatesh, Karnataka (IN); Venkatesh Laxminarayan Hegde, Karnataka (IN)

Correspondence Address: FOLEY AND LARDNER, SUITE 500, 3000 K STREET NW, WASHINGTON, DC, 20007, US

(73) Assignee: COUNCIL OF SCIENTIFIC AND INDUSTRIAL RESEARCH

(21) Appl. No.: 10/400,919

(22) Filed: Mar. 28, 2003

(51) Int. Cl. A61K 39/395; C12Q 1/70; C12P 21/04; C07K 14/47; C07K 16/18

(52) U.S. Cl. 424/130.1; 435/70.21; 530/410; 530/388.1

(57) ABSTRACT

The present invention provides a hapten-carrier conjugate as antigen having haptenic mannitol epitopes, the invention also provides a method of preparing hapten-carrier conjugate as immunogen possessing mannitol groups or epitopes to raise antibodies specific to D-mannitol and the present invention further provides a use of hapten-carrier conjugate as an antigen to detect D-Mannitol specific human IgE.

### FIELD OF THE INVENTION

The present invention relates to a hapten-carrier conjugate as antigen having haptenic mannitol epitopes. The invention also relates to a method of preparing hapten-carrier conjugate as immunogen possessing mannitol groups or epitopes to raise antibodies specific to D-mannitol. The present invention further relates to use of hapten-carrier conjugate as an antigen to detect D-Mannitol specific human IgE.

### BACKGROUND ART

D-Mannitol (hereafter referred to as mannitol) is a sugar alcohol or polyol, which is widely used as a food and drug additive owing to its chemical inertness, non-hygroscopicity, sweetness and non-toxicity. Mannitol is the excipient of choice in pharmaceutical preparations because of its aforementioned unique functional properties. It is about 50% as sweet as sucrose and has a desirable cooling effect, a property utilized to mask bitterness in pharmaceuticals. Mannitol is a low calorie sweetener and non-carcinogenic. Because of its osmotic properties, mannitol promotes the movement of fluid from the intracellular into extra cellular space. Hence, mannitol in the form of 20% IV (intravenous) infusions is used as a therapeutic agent in many clinical situations such as drug intoxication, oliguric renal failure, glaucoma, cerebral edema, and increased intracranial pressure. Hypersensitivity reactions to IV infusion of mannitol in humans have been reported in the medical literature (Spaeth G L et al. Arch Ophthal 1967;78:583-584; Lamb J D, Keogh J A M Canad Anaesth Soc J 1979;26:435-436; Findlay S R et al. J Allergy Clin Immunol 1984;73:578-583; McNeill I Y Drug Intell Clin Pharm 1985;19:552-553; Ackland S P, Hillcoat B L Cancer Treat Rep 1985;69:562-563; Biro P et al. Anaesthesist 1992;41:130-133; Schmid P, Wüthrich B Allergy 1992;47:61-62). Recently, we reported a case of anaphylaxis caused by ingestion of minute amounts of mannitol present naturally in some foods such as pomegranate and mushroom (Hegde V L et al. Allergy Clin Immunol Int 2002;14:37-39; Hegde V L et al. Allergology Int 2002;51:121-129).

The property of chemical inertness of mannitol (i.e., not having any reactive groups) that qualifies it as an excellent food and drug additive also makes it extremely difficult to couple to macromolecular carriers in order to obtain effective hapten-carrier conjugate to raise hapten-specific antibodies. Therefore, an indirect method should be used to obtain mannitol groups or epitopes on carrier proteins. Mannitol groups or epitopes can be obtained on carrier proteins by the reaction of amino groups of carriers with D-mannose by reductive alkylation reaction (also referred to as reductive amination). This is based on the following observations: Schwartz B A and Gray G R, Arch Biochem Biophys 1977;181:542-549 and Gray G R, Methods Enzymol 1978;55:155-160 used reductive amination in the presence of sodium cyanoborohydride to attach disaccharides to proteins to raise antibodies directed against terminal (non-reducing) cyclic monosaccharide units.

Using a similar approach, Walton D J et al., Carbohydr Res 1984;128:37-49, synthesized N-(1-deoxy-hexitol-1-yl) derivatives of L-valine, L-alanine, L-threonine, and L-leucine, which served as reference compounds for the identification of nonenzymically glycated proteins formed in diabetes.

U.S. Pat. No. 5,484,735 (Davis L E, Anderson B E 1996) and PCT No. WO 91/02978 (Anderson B E, Morton G, Davis L E 1991) describe, using a similar method, raising antibodies directed to Glc-ol-X, where Glc-ol is the reduced form of a sugar attached to the alpha amino group of X, and X is the N-terminal amino acid of a glycosylated protein, except that X cannot be lysine, and the carrier is an immunogenic compound other than the glycosylated protein. U.S. Pat. No. 4,797,473 (Tarsio J F and Furcht L T 1989) provides a hybridoma which yields a monoclonal antibody that binds to an epitope on an unreduced, non-enzymatically-glycated plasma protein, and which is substantially free of cross-reactivity with the corresponding non-glycated plasma protein.

Myint T et al., Biochim Biophys Acta 1995;1272:73-79, teaches the development of polyclonal IgG antibodies in rabbits specific for the Amadori compound, a product of an early stage of non-enzymic glycation in vivo, by immunization with hexitol-lysine coupled to various proteins. The antibody was used to specifically detect glycated proteins (Amadori products) in various tissues of streptozotocin-induced diabetic rats. Polyclonal and monoclonal antibodies have been raised against reduced glycated human plasma lipoproteins, and these antibodies do not recognize nonglycosylated or unreduced glycosylated lipoproteins. The dominant epitope in these cases was found to be glucitol-lysine (Curtiss L K and Witztum J L, J Clin Invest 1983;72:1427-1438; Witztum J L et al., Proc Natl Acad Sci USA 1983;80:2757-2761).

These and related prior arts focus on developing immunoassays by raising specific antibodies to detect and quantitate various non-enzymically glycated proteins in serum and different tissues formed as a result of high blood glucose concentration under diabetic conditions.

In view of the above discussion, it was felt necessary to prepare hapten-carrier conjugates possessing mannitol groups or epitopes to detect mannitol-specific antibodies by in vitro and in vivo diagnostic tests in cases of hypersensitivity to mannitol, and to raise specific antibodies to mannitol in laboratory animals, which are essential in developing immunoassays.

### References Cited

US Patent Documents

| | | | |
|---|---|---|---|
| 4797473 | Jan., 1989 | Tarsio et al. | 435/7.95 |
| 5484735 | Jan., 1996 | Davis et al. | 436/538 |

### Foreign Patent Documents

WO 91/02978 March 1991 Anderson et al. GO1N 33/535

### Other References

Ackland S P, Hillcoat B L. Cancer Treat Rep 1985;69:562-563.

Biro P, Schmid P, Wüithrich B. Anaesthesist 1992;41:130-133.

Curtiss L K, Witztum J L. J Clin Invest 1983;72:1427-1438.

Findlay S R, Kagey-Sobotka A, Lichtenstein L M. J Allergy Clin Immunol 1984;73:578-583.

Gray G R. Methods Enzymol 1978;55:155-160.

Hegde V L, Mahesh P A, Venkatesh Y P. Allergy Clin Immunol Int 2002;14:37-39.

Hegde V L, Das J R, Venkatesh Y P. Allergology Int 2002;51:121-129.

Lamb J D, Keogh J A M. Canad Anaesth Soc J 1979;26:435-436.

McNeill I Y. Drug Intell Clin Pharm 1985;19:552-553.

Myint T, Hoshi S, Ookawara T, Miyazawa N, Suzuki K, Taniguchi N. Biochim Biophys Acta 1995;1272:73-79.

Roy R, Katzenellenbogen E, Jennings H J. Can J Biochem Cell Biol 1984;62:270-275.

Sashidhar B R, Capoor A K, Ramana D. J Immunol Methods 1994;167:121-127.

Schmid P, Wüithrich B. Allergy 1992;47:61-62.

Schwartz B A, Gray G R. Arch Biochem Biophys 1977;181:542-549.

Spaeth G L, Spaeth E B, Spaeth P G, Lucier A C. Arch Ophthal 1967;78:583-584.

Spencer N. J Chromatogr 1967;30:566-571.

Stults N L, Asta L M, Lee Y C. Anal Biochem 1989;180:114-119.

Walton D J, Ison E R, Szarek W A. Carbohydr Res 1984;128:37-49.

Witztum J L Steinbrecher U P, Fisher M, Kesaniemi A. Proc Natl Acad Sci USA 1983;80:2757-2761.

### OBJECTS OF THE INVENTION

The main object of the present invention is to provide an antigen having haptenic mannitol epitopes.

An object of the present invention is to provide an antigen said antigen with hapten conjugate to detect mannitol-specific antibodies.

Yet another object of the present invention, is to provide a method for the preparation of antibodies specific to D-mannitol prepared by immunizing an animal with a hapten-carrier conjugate as an antigen, which has D-mannitol epitopes.

Another object of the present invention is to provide an affinity-purified antibody showing specificity for D-mannitol, and was substantially free of cross-reactivity towards other structurally related compounds.

Yet another object of the present invention is to provide mannitol-carrier conjugates used as utility reagents to demonstrate cell-bound mannitol-specific IgE by skin prick test (an in vivo diagnostic test) in an allergic subject sensitized to mannitol.

### SUMMARY OF THE INVENTION

The present invention provides a hapten-carrier conjugate as antigen having haptenic mannitol epitopes. The invention also provides a method of preparing hapten-carrier conjugate as immunogen possessing mannitol groups or epitopes to raise antibodies specific to D-mannitol. The present invention further relates to use of hapten-carrier conjugate as an antigen to detect D-Mannitol specific human IgE. Detailed description of the invention.

### DETAILED DESCRIPTION OF THE INVENTION

Accordingly, the present invention provides a hapten-carrier conjugate as antigen having haptenic mannitol epitopes, said antigen comprising hapten conjugate of Formula I

N-(1-deoxy-D-mannitol-1-yl)ₙ-carrier Formula I

wherein,

‘N’=both N^{ε} of lysine residues and N^{α} of the N-terminal residue of the carrier

‘n’=the average number of amino groups modified by conjugation.

An embodiment of the present invention, wherein the carrier is selected from any immunogenic protein.

Another embodiment of the present invention, wherein the carrier is bovine serum albumin (BSA).

Still another embodiment of the present invention, wherein the value of n is in the range of 25-30.

Yet another embodiment of the present invention, wherein the modification of amino groups on the carrier protein is determined by the number of unmodified amino groups present on the carrier, by trinitrobenzene sulfonic acid (TNBS) assay. The present invention also provides a method of raising and purifying antibodies specific to D-mannitol, said method comprising the steps of:

(a) immunizing a mammal with, a hapten-carrier conjugate carrier as an antigen having haptenic mannitol epitopes obtained by reductive alkylation of carrier protein with sugar D-mannose, and having a general formula I

N-(1-deoxy-D-mannitol-1-yl)ₙ-carrier Formula I

wherein,

‘N’=both N^{ε} of lysine residues and N^{α} of the N-terminal residue of the carrier

‘n’=the average number of amino groups modified by conjugation.

(b) extracting the antiserum from the immunized animals; and

(c) purifying D-mannitol-specific antibodies from the antiserum by affinity chromatography by preparing the hapten affinity matrix by immobilizing the D-mannitol-carrier 2 conjugate on to oxidized Sepharose CL-6B to obtain purified antibodies.

An embodiment of the present invention, a method wherein carrier for step (a) is an immunogenic protein.

Another embodiment of the present invention, a method wherein carrier for step (a) is bovine serum albumin (BSA).

Still another embodiment of the present invention, a method wherein the average number of amino groups modified by conjugation is determined by the number of un-modified amino groups on the carrier protein, by trinitrobenzenesulfonic acid (TNBS) assay.

Yet another embodiment of the present invention, a method wherein n having values

ranging from 25 to 30 of available coupling sites on the carrier.

It is also an embodiment of the present invention, a method wherein the sugar is selected from any reducing monosaccharide.

Still another embodiment of the present invention, a method the conjugate having sugar alcohol epitope.

Further embodiment of the present invention, a method wherein quantitative spectrophotometer assay is adopted for using chromotropic acid reagent to test the conjugates for the formation of sugar alcohol groups or epitopes.

Yet another embodiment of the present invention, a method wherein the mammal is selected preferably from White New Zealand rabbit or mouse or rat.

Still another embodiment of the present invention, a method wherein in step (c) the carrier2 is keyhole limpet hemocyanin (KLH).

Further embodiment of the present invention, a method wherein about 50% of free amino groups of the KLH are modified by conjugation and the rest are available for immobilizing the conjugate on to oxidized Sepharose CL-6B matrix.

The present invention also provides a use, wherein antibody specific to D-mannitol as a reagent for in vivo diagnosis in mammals of hypersensitivity to D-mannitol by skin prick test (SPT) to indicate the presence of cell-bound mannitol-specific IgE. An embodiment of the present invention, a use wherein the conjugate is used as a reagent in the detection of mannitol-specific IgE (Immunoglobulin E) in the serum of allergic subjects who have hypersensitivity to mannitol.

Another embodiment of the present invention, a use wherein the presence of serum mannitol-specific IgE is detected in this in vitro diagnostic test.

Yet another embodiment of the present invention, a use wherein the carrier human serum albumin (HSA) as a reagent for specific immunotherapy or desensitization in allergic subjects sensitized to mannitol.

Still another embodiment of the present invention, a use wherein affinity-purified antibody displaying specificity for D-mannitol, and is substantially free of cross-reactivity towards other structurally related compounds.

Yet another embodiment of the present invention, a use wherein the mannitol-carrier conjugates are also used as utility reagents to demonstrate cell-bound mannitol-specific IgE by skin prick test (an in vivo diagnostic test) in an allergic subject sensitized to mannitol.

The invention is further explained in the form of following embodiments.

The immunogen used to raise specific antibodies in the present invention comprises sugar alcohol (D-mannitol) groups or epitopes on an immunogenic carrier protein, bovine serum albumin (BSA) obtained by coupling reducing sugar (D-mannose) by the method of reductive alkylation (also called reductive amination) in the presence of sodium cyanoborohydride. The conjugate possessing approximately 50% of its free amino groups (25-30 groups) modified, which resulted in D-mannitol epitopes, was used for immunization.

The reducing sugar (D-mannose) was coupled similarly to a different carrier, keyhole limpet hemocyanin (KLH), and this hapten-carrier conjugate was immobilized on to oxidized Sepharose CL-6B in the presence of borane-pyridine complex. This affinity matrix (mannitol-KLH-Sepharose CL-6B) was used to purify antibodies specifically directed against the D-mannitol epitope.

In another embodiment of the present invention, a method to test the conjugates for the formation of sugar alcohol groups or epitopes by a qualitative color reaction was developed, based on the known principle of quantitative spectrophotometric assay for sugar alcohols or polyols using chromotropic acid reagent (Spencer N. J Chromatogr 1967;30:566-571).

The specificity of the purified antibody towards free D-mannitol, and its cross-reactivity towards other structurally related compounds (sugars and sugar alcohols), are determined by ELISA inhibition experiments.

In yet another embodiment of the present invention, the D-mannitol-BSA conjugate was used to demonstrate cell-bound mannitol-specific human IgE by skin prick test in an allergic subject sensitized to mannitol.

### BRIEF DESCRIPTION OF THE ACCOMPANIED DIAGRAMS

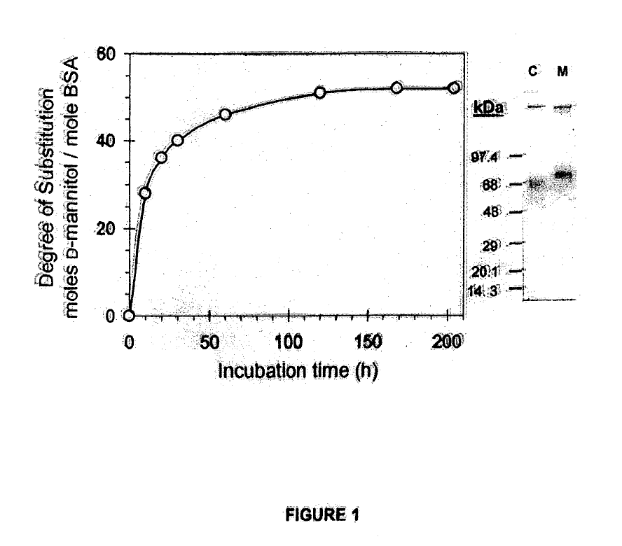

FIG. 1 depicts reductive amination of D-mannose with bovine serum albumin (BSA) to obtain D-mannitol-BSA conjugate. The reaction was monitored by TNBS assay for amino groups. Degree of substitution was calculated from the decrease in amino groups due to conjugation. The photograph on the right side shows the SDS-PAGE (10% gel, under reducing conditions) pattern of control BSA (C) and mannitol-BSA (M). The mannitol-BSA sample had been reductively aminated for 204 h. Increase in molecular weight of ˜9 kDa in mannitol-BSA conjugate, in comparison with control BSA, was observed, which corresponded to an addition of ˜50 groups of mannitol.

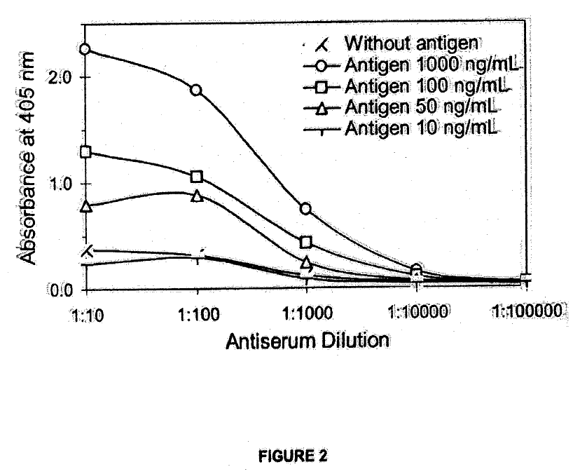

FIG. 2 depicts titration of D-mannitol-BSA rabbit antiserum at different coated antigen concentrations. D-Mannitol-KLH conjugate was used as the coating antigen to specifically detect D-mannitol-specific antibodies.

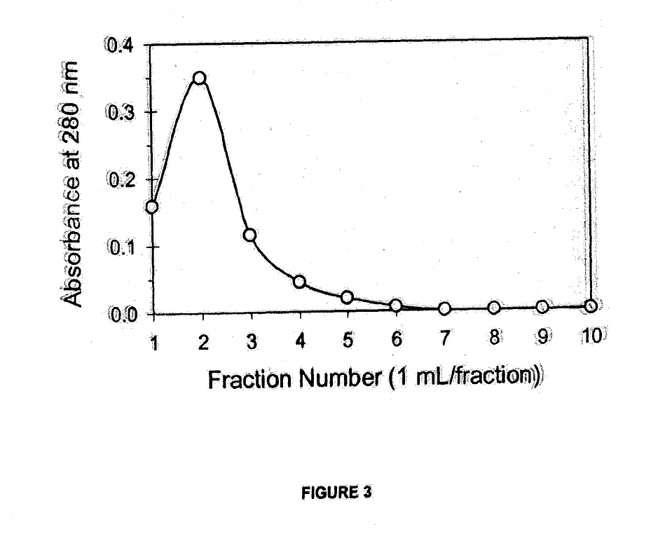

FIG. 3 depicts hapten affinity purification of D-mannitol-specific antibody on D-mannitol-KLH-Sepharose CL-6B column (0.8 cm id×4 cm). Six mL of rabbit antiserum diluted 1:1 in PBS was passed through the column, the flow-through was recycled twice, followed by washing with 50 mL of PBS. The figure shows the typical elution profile of bound antibody using 0.1 M glycine-HCl buffer, pH 2.9.

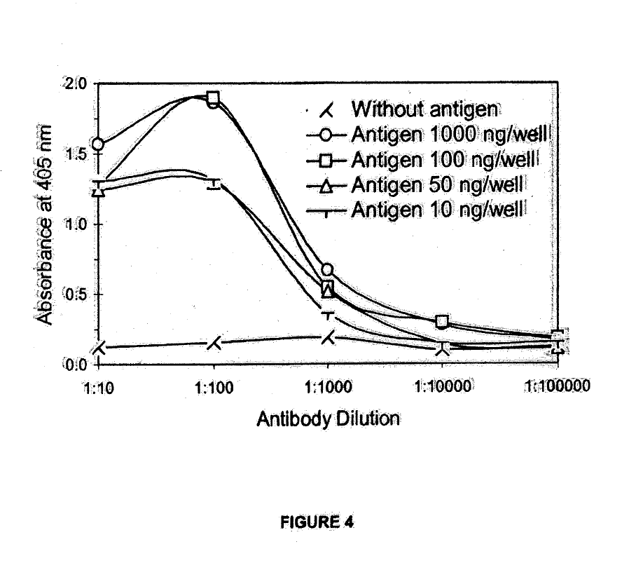

FIG. 4 depicts binding curves obtained with hapten-affinity purified D-mannitol-specific antibody at various dilutions and at different coated antigen (D-Mannitol-KLH conjugate) concentrations.

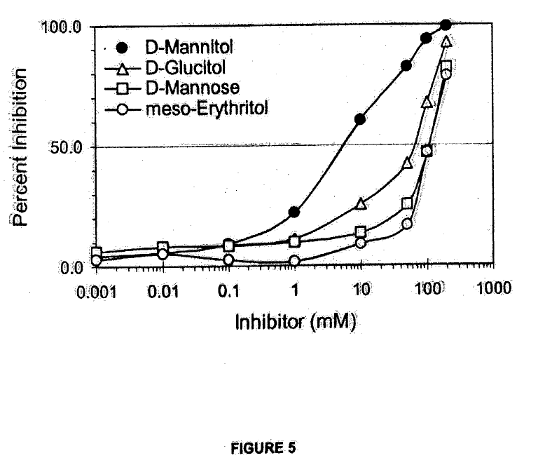

FIG. 5 depicts specificity of the purified D-mannitol-specific antibody determined by ELISA inhibition. D-Mannitol-KLH conjugate (10 ng/well) was used for coating. The purified antibody (at 1:100 dilution) was preincubated with various inhibitors at different concentrations at 37° C. for 1 h before adding to the coated wells. The antibody showed maximum inhibition with D-mannitol. The concentration of each inhibitor required for 50% inhibition (IC50) was obtained from this graph (D-mannitol, 5.25 mM; D-glucitol (sorbitol), 60 mM; D-mannose, 108 mM; meso-erythritol, 108 mM), and percent cross-reactivity was calculated (see Table 1).

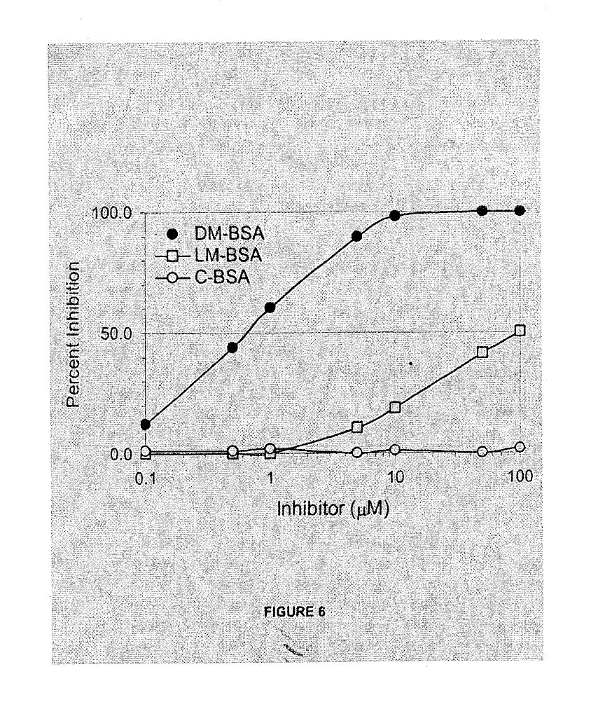

FIG. 6 depicts stereo specificity of the purified D-mannitol-specific antibody determined by ELISA inhibition. As L-mannitol is not available commercially, L-mannitol-BSA conjugate (prepared using L-mannose) was used as inhibitor and the inhibition was compared with that of D-mannitol-BSA conjugate. The IC50 values were, 0.64 μM for D-mannitol-BSA conjugate, and 95 μM for L-mannitol-BSA conjugate. Percent cross-reactivity is shown in Table 1. The cross-reactivity with L-mannitol-BSA conjugate was <1%.

The invention is further illustrated in the form of following examples. However, these examples shall not be construed as limiting the scope of the invention.

### EXAMPLE 1

Hapten-carrier conjugates: Coupling of D-mannose to BSA to obtain mannitol epitopes D-Mannose was conjugated to a carrier protein, bovine serum albumin (BSA) by reductive amination (also termed reductive alkylation) as described by Roy R et al., Can J Biochem Cell Biol 1984;62:270-275. BSA (68 mg, 1 μmol), D-mannose (100 mg, 282 μmol), sodium cyanoborohydride (100 mg, 1.59 mmol) were dissolved in 5.0 mL of 0.2 M borate buffer (pH 8.0), and incubated at 37° C. Aliquots of the reaction mixture were withdrawn at various time intervals, reaction was stopped by bringing the pH down to 3.5-4.0 by adding 80% acetic acid, dialyzed extensively against phosphate-buffered saline (PBS) at 4° C. and analyzed for decrease in amino groups to determine degree of substitution. As a control, BSA was incubated as above, excluding D-mannose (control BSA). Similarly, D-mannose was conjugated to KLH, and L-mannose to BSA. The conjugates were sterile filtered and stored below −20° C. TNBS assay for amino groups

Amino groups in the control and conjugates were determined using trinitrobenzenesulfonic acid (TNBS) reaction (Sashidhar B R et al., J Immunol Methods 1994;167:121-127). To 1 mL of control or conjugated BSA (100 μg) solution, were added 1 mL of 4% NaHCO₃ (pH 8.5) and 1 mL of 0.01% TNBS solution prepared freshly in water. The reaction was carried out at 42° ±2° C. for 2 h, followed by the addition of 1 mL 10% SDS and 0.5 mL of 1 N HCl. The absorbance was read at 335 nm in a spectrophotometer against an appropriate blank.

An average of 52 of 61 available amino groups on BSA were modified by conjugation at the end of 204 h of incubation (see FIG. 1). About 50% of the amino groups (25-30) were modified at the end of 10 h. SDS-PAGE analysis of the conjugate (obtained at 204 h incubation) in comparison with control BSA showed an increase in molecular weight of 9 kDa corresponding to an addition of about 52 mannitol groups, which agrees well with that obtained by TNBS assay.

### Qualitative Color Test to Confirm the Formation of Sugar Alcohol Epitopes on Carrier

The spectrophotometric assay specific for polyols described by Spencer N, J Chromatogr 1967;30:566-571 was employed with modification as a qualitative color test to confirm the formation of sugar alcohol epitopes on carrier protein. Briefly, to 0.2 mL samples were added 0.05 mL of 0.01 M sodium periodate in 1 N sulfuric acid. After mixing and allowing the solutions to stand at 25° C. for 10 min, 0.02 mL of 10% w/v sodium bisulfite was added with immediate mixing followed by 0.02 mL of 2% aqueous chromotropic acid solution. Finally, 0.3 mL concentrated sulfuric acid was added and vortexed. A positive test is indicated by the development of violet color. However, this test could not be used here as a quantitative assay since the reaction mixture turned opaque due to protein precipitation during the test. The color test was positive for D-mannitol, mannitol-BSA, mannitol-KLH and was negative for D-mannose, BSA, KLH, thus confirming the formation of mannitol epitopes in mannitol-BSA and mannitol-KLH conjugates.

### Immunization of Animal and Collection of Blood

Male White New Zealand rabbit 7-months-old housed in Animal House facility of our institute was used for the experiment, according to standard operating procedures, after obtaining approval from the Institute Animal Ethics committee (IAEC). The first injection comprising D-mannitol-BSA conjugate (0.5 mL, 1 mg protein) micro emulsified with 0.5 mL of 3× concentrated Freund's complete adjuvant was administered intraderm-ally at 8-10 sites on the back of the animal. After 4 weeks, booster doses containing 0.5 mg of the antigen micro emulsified with Freund's incomplete adjuvant were given by intramuscular injection at 15 days intervals. The animal was bled by marginal ear vein puncture 1 week before the first injection (to obtain pre-immune serum) and 1 week after each booster dose. The pooled serum was stored below −20° C.

### Titration of Antiserum

To check for the formation of hapten-specific antibodies the antiserum was analyzed by non-competitive ELISA using the coated antigen format. The titer of the antiserum was determined by measuring the binding of serial dilutions of antiserum to coated mannitol-KLH conjugate. Flat bottom polystyrene microtiter wells were coated with mannitol-KLH conjugate at 1000, 100, 50, 10, and 1 ng per well in carbonate-bicarbonate buffer, pH 9.6 by incubating at 4° C. overnight. The wells were washed thrice between steps using PBS containing 0.05% Tween 20 (PBS-T). Blocking was done using 0.5% gelatin in PBS-T (blocking buffer) at 37° C. for 30 min. Antiserum diluted in blocking buffer (1:10, 1:100, 1:1000, 1:10000, 1:25000, 1:50000, 1:75000; 1:100000, 1:200000, 1:400000) was added (100 μL/well) and incubated at 37° C. for 2 h. Mouse anti-rabbit IgG-alkaline phoshatase secondary antibody (1:5000 dilution in blocking buffer, 100 μL/well) was added and incubated at 37° C. for 1 h. Color development was done using p-nitrophenyl phosphate (1 mg/mL, 100 μL/well) in diethanolamine buffer, pH 9.8, at 37° C. for 30 min. The reaction was stopped by adding 3 M NaOH (40 μL/well) and the absorbance at 405 nm was read in an ELISA reader.

The titration of antiserum with mannitol-KLH conjugate carried out by non-competitive ELISA is shown in FIG. 2.

### Preparation of D-mannitol-KLH-Sepharose CL-6B Affinity Column

D-Mannitol-KLH conjugate (hereafter referred to as mannitol-KLH) with 50% modification of amino groups was coupled to Sepharose CL-6B according to the method of Stults N L et al., Anal Biochem 1989;180:114-119 with slight modification. The gel was first washed thoroughly with water to remove preservative, and then suspended in water to obtain 0.2 g moist gel/mL (total 10 mL). The gel was then oxidized by adding solid sodium periodate (NaIO₄) to a final concentration of 25 mM. The mixture was swirled at room temperature (25° C.) for 30 min; unreacted NaIO₄ was consumed by the addition of an equimolar amount of ethylene glycol. After 15 min, the gel was thoroughly washed with water followed by 0.2 M phosphate buffer, pH 7.0. The moist gel (0.2 g/mL suspension) in phosphate buffer was mixed with mannitol-KLH (2 mg) followed by direct addition of borane-pyridine complex to a final concentration of 25 mM. The reaction mixture was agitated first at 25° C. for 3 h and then continued at 4° C. for 10 h. Next, the gel beads were extensively washed with water to remove unreacted borane-pyridine complex and protein conjugate. The gel was finally packed into a glass column (0.8 cm id×4 cm), and equilibrated with 20 mM sodium phosphate buffer, pH 7.4 containing 140 mM NaCl, and stored in the same buffer with 0.01% merthiolate as a preservative.

### Affinity Purification of Mannitol-Specific Antibody on D-mannitol-KLH-Sepharose CL-6B Column

Antiserum was diluted 1:1 in PBS and passed through the hapten affinity column. The flow-through was recycled twice through the column. After washing with PBS, the bound antibody was eluted with 0.1 M glycine-HCl buffer, pH 2.9. One mL fractions were collected and the absorbance was monitored at 280 nm. The fractions containing protein were pooled and stored in aliquots below −20° C.

The elution of bound antibody (after loading and washing) is presented in FIG. 3. Most of the antibody was found to elute in the first 3-4 fractions. The specific antibody yield was calculated based on the reference value of A^{0.1%}_{1 cm} (at 280 nm) of 1.40 for rabbit IgG. The total absorbance of the first three fractions was 0.623, and the mannitol-specific antibody yield was 0.15 mg/mL rabbit antiserum.

The hapten affinity-purified D-mannitol-specific antibody was used for ELISA experiments to obtain the binding curves using D-mannitol-KLH conjugate for coating, and to study the specificity and cross-reactivity by ELISA inhibition experiments. Here, purified antibody was preincubated with various concentrations of inhibitors at 37° C. for 1 h before adding to coated microtiter wells. Other steps were as described under ‘Titration of antiserum’. Percent cross-reactivity for various inhibitors was calculated using the formula:

(IC50 for D-mannitol÷IC50 for test compound)×100

where, IC50 is the concentration required for 50% inhibition in ELISA.

In the ELISA inhibition experiment, D-mannitol showed maximum inhibition (FIG. 4). Among structurally related compounds, inhibition by D-sorbitol was higher compared to that by meso-erythritol and D-mannose. The cross-reactivity of D-mannitol-specific antibody for these compounds was less than 5%, and in the case of D-glucitol (sorbitol) the cross-reactivity was ˜9%. The cross-reactivity with L-mannitol-BSA conjugate was <1% (Table 1). Inhibition using L-lysine showed a cross-reactivity of 2%.

### EXAMPLE 2

A D-mannitol-BSA and D-mannitol-KLH conjugate was used for skin prick test (SPT) in a mannitol-allergic subject, after obtaining Institutional Ethics Committee (IEC) clearance, and informed consent. D-Mannitol and its conjugates with BSA and KLH produced positive test whereas, D-mannose and control BSA and control KLH gave negative test (Table 2). L-Mannitol BSA conjugate was negative in SPT. All these samples were negative in SPT in 10 normal subjects. These results indicated the presence of mannitol-specific mast cell-bound human IgE in the allergic subject sensitized to mannitol.

TABLE 1
Specificity of hapten affinity-purified
mannitol-specific antibody

| | | | Percent |
|---|---|---|---|
| Competitor | IC50 | | cross-reactivity |
| D-Mannitol | 5.25 | mM | 100.0 |
| D-Glucitol | 60.00 | mM | 8.8 |
| D-Mannose | 108.00 | mM | 4.9 |
| meso-Erythritol | 108.00 | mM | 4.9 |
| D-Mannitol-BSA conjugate | 0.64 | μM | 100.00 |
| L-Mannitol-BSA conjugate | 95.00 | μM | 0.67 |

TABLE 2
Skin prick test (SPT) in an allergic
subject sensitized to mannitol

| Sample | Wheal/flare diameter (mm) |
|---|---|
| Negative control (glycerinated PBS) | 1/0 |
| Positive control (Histamine base, 0.1%) | 6/30 |
| D-Mannitol, 0.1% | 5/25 |
| D-Mannose, 0.1% | 1/0 |
| D-Mannitol-BSA conjugate | 4/30 |
| D-Mannitol-KLH conjugate | 4/25 |
| L-Mannitol-BSA conjugate | 1/0 |
| Control BSA | 1/0 |
| Control KLH | 1/0 |

We claim

1. A hapten-carrier conjugate as antigen having haptenic mannitol epitopes, said antigen comprising hapten conjugate of Formula I
N-(1-deoxy-D-mannitol-1-yl)ₙ-carrier Formula I
wherein,
‘N’=both N^{ε} of lysine residues and N^{α} of the N-terminal residue of the carrier
‘n’=the average number of amino groups modified by conjugation.

2. The antigen of claim 1, wherein the carrier is selected from any immunogenic protein.

3. The antigen of claim 1, wherein the carrier is bovine serum albumin (BSA).

4. The antigen of claim 1, wherein the value of n is in the range of 25-30.

5. The antigen of claim 1, wherein the modification of amino groups on the carrier protein is determined by the number of unmodified amino groups present on the carrier, by trinitrobenzene sulfonic acid (TNBS) assay.

6. A method for raising and purifying antibodies specific to D-mannitol, said method comprising the steps of:
a. immunizing a mammal with, a hapten-carrier conjugate carrier as an antigen having haptenic mannitol epitopes obtained by reductive alkylation of carrier protein with sugar D-mannose, and having a general formula I
N-(1-deoxy-D-mannitol-1-yl)ₙ-carrier Formula I
wherein,
‘N’=both N^{ε} of lysine residues and N^{α} of the N-terminal residue of the carrier
‘n’=the average number of amino groups modified by conjugation.
b. extracting the antiserum from the immunized animals; and
c. purifying D-mannitol-specific antibodies from the antiserum by affinity chromatography by preparing the hapten affinity matrix by immobilizing the D-mannitol-carrier 2 conjugate on to oxidized Sepharose CL-6B to obtain purified antibodies.

7. The method of claim 6, wherein carrier for step (a) is an immunogenic protein.

8. The method of claim 6, wherein carrier for step (a) is bovine serum albumin (BSA).

9. The method of claim 6, wherein the average number of amino groups modified by conjugation is determined by the number of unmodified amino groups on the carrier protein, by trinitrobenzenesulfonic acid (TNBS) assay.

10. The method of claim 6, wherein n is from 25 to 30 of available coupling sites on the carrier.

11. The method of claim 6, wherein the sugar is selected from any reducing monosaccharide.

12. The method of claim 6, wherein the conjugate having sugar alcohol epitope.

13. The method of claim 6, wherein quantitative spectrophotometer assay is adopted for using chromotropic acid reagent to test the conjugates for the formation of sugar alcohol groups or epitopes.

14. The method of claim 6, wherein the mammal is selected preferably from White New Zealand rabbit or mouse or rat.

15. The method of claim 6, wherein in step (c) the carrier2 is keyhole limpet hemocyanin (KLH).

16. The method of claim 6, wherein about 50% of free amino groups of the KLH are modified by conjugation and the rest are available for immobilizing the conjugate on to oxidized Sepharose CL-6B matrix.

17. The use of antibody specific to D-mannitol as a reagent for in vivo diagnosis in mammals of hypersensitivity to D-mannitol by skin prick test (SPT) to indicate the presence of cell-bound mannitol-specific IgE.

18. The use as stated in 17, wherein the conjugate is used as a reagent in the detection of mannitol-specific IgE (Immunoglobulin E) in the serum of allergic subjects who have hypersensitivity to mannitol.

19. The use as stated in claim 17, wherein the presence of serum mannitol-specific IgE is detected in this in vitro diagnostic test.

20. The use as stated in claim 17, wherein the carrier human serum albumin (HSA) as a reagent for specific immunotherapy or desensitization in allergic subjects sensitized to mannitol.

21. The use as stated in claim 17, wherein affinity-purified antibody displaying specificity for D-mannitol, and is substantially free of cross-reactivity towards other structurally related compounds.

22. The use as stated in claim 17, wherein the mannitol-carrier conjugates are also used as utility reagents to demonstrate cell-bound mannitol-specific IgE by skin prick test (an in vivo diagnostic test) in an allergic subject sensitized to mannitol.
